(19) United States
(12) Patent Application Publication
(10) Pub. No.: US 2003/0017208 A1
(43) Pub. Date: Jan. 23, 2003

(54) Electrospun pharmaceutical compositions

(76) Inventors: Francis Ignatious, Exton, PA (US); John M. Baldoni, Glenmoore, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/181,640

(22) PCT Filed: Jan. 25, 2001
(86) PCT No.: PCT/US01/02399

(51) Int. Cl. A61K 9/14

(52) U.S. Cl. 424/486

(57) ABSTRACT

The present invention is directed to an electrospun pharmaceutical composition comprising a pharmaceutically acceptable actibe agent, and a pharmaceutically acceptable polymeric carrier for use in therapy.

### FIELD OF THE INVENTION

This invention relates to nanofibers of drug particles, method of preparation thereof and pharmaceutical compositions containing these nanofibers. This invention further relates to the use of such nanofibers in designing various dosage forms to achieve maximum bioavailability of a drug moiety.

### BACKGROUND OF THE INVENTION

It is known that the rate of dissolution of a particulate drug can increase with increasing surface area, i.e., decreasing particle size. Consequently, methods of making finely divided drugs have been studied and efforts have been made to control the size and size range of drug particles in pharmaceutical compositions. For example, dry milling techniques have been used to reduce particle size and hence influence drug absorption. However, in conventional dry milling, as discussed by Lachman, et al., The Theory and Practice of Industrial Pharmacy, Chapter 2, “Milling”, p. 45, (1986), the limit of fineness is reached in the region of 100 microns (100,000 nm) when material cakes on the milling chamber. Lachman, et al. note that wet grinding is beneficial in further reducing particle size, but that flocculation restricts the lower particle size limit to approximately 10 microns (10,000 nm). However, there tends to be a bias in the pharmaceutical art against wet milling due to concerns associated with contamination. Commercial airjet milling techniques have provided particles ranging in average particle size from as low as about 1 to 50 micrometers (1,000-50,000 nm).

Other techniques for preparing pharmaceutical compositions include loading drugs into liposomes or polymers, e.g., during emulsion polymerization. However, such techniques have problems and limitations. For example, a lipid soluble drug is often required in preparing suitable liposomes. Further, unacceptably large amounts of the liposome or polymer are often required to prepare unit drug doses. Further still, techniques for preparing such pharmaceutical compositions tend to be complex. A principal technical difficulty encountered with emulsion polymerization is the removal of contaminants, such as unreacted monomer or initiator, which can be toxic, at the end of the manufacturing process.

U.S. Pat. No. 4,540,602 (Motoyama et al.) discloses a solid drug pulverized in an aqueous solution of a water-soluble high molecular substance using a wet grinding machine. However, Motoyama et al. teach that as a result of such wet grinding, the drug is formed into finely divided particles ranging from 0.5.mu.m (500 nm) or less to 5.mu.m (5,000 nm) in diameter.

U.S. Pat. No. 5,145,684 (Liversidge et al) discloses dispersible crystalline drug substances having particle sizes lower than 400 nm, for increased bioavailability, produced by wet milling.

EPO 275,796 describes the production of colloidal dispersible systems comprising a substance in the form of spherical particles smaller than 500 nm. However, the method involves a precipitation effected by mixing a solution of the substance and a miscible non-solvent for the substance and results in the formation of non-crystalline nanoparticle. Furthermore, precipitation techniques for preparing particles tend to provide particles contaminated with solvents. Such solvents are often toxic and can be very difficult, if not impossible, to adequately remove to pharmaceutically acceptable levels to be practical.

U.S. Pat. No. 4,107,288 describes particles in the size range from 10 to 1,000 nm containing a biologically or pharmacodynamically active material. However, the particles comprise a crosslinked matrix of macromolecules having the active material supported on or incorporated into the matrix.

Solid dispersions of drugs in polymers are being investigated to address the diminished bioavailability of poorly water soluble drugs. For a recent review see Serajuddin, Journal of Pharmaceutical sciences, 1999, 88(10), 1058.

An area of great interest is the rapid dissolve dosage forms, which is targeted towards the specific needs of pediatric, geriatrics and patients with dysphagia.

U.S. Pat. No. 4,855,326 describes a melt spinnable carrier agent such as sugar is combined with a medicament then converted into fiber form by melt spinning with “cotton candy” fabricating equipment. The as-spun product is converted to compacted individual dosage units. For certain medicaments a binding agent is added to the carrier agent. Examples are presented for oral administration, topical application, systemic and non-systemic, intravenous and intramuscular infusion via multicameral containers. All applications utilize the extraordinarily rapid entry into solution upon contact with a solvent.

U.S. Pat. Nos. 4,946,684; 5,298,261; 5,466,464; 5,501,861; 5,762,961; 5,866,163 disclose taste masked rapidly dissolving dosage forms having organoleptically acceptable properties disintegrate rapidly in patients mouth without chewing or with minimum amount of water.

U.S. Pat. No. 5,948,430 discloses a polymeric film composition providing instant wetability followed by rapid dissolution/disintegration upon administration in the oral cavity. This may be applicable only to soluble drugs.

Pulmonary delivery, both as immediate and modified release, dosage forms are being actively investigated.

U.S. Pat. No. 5,747,001 discloses the advantages of aerosolized nanoparticles in pulmonary delivery.

WO 99/48476 describes the use of drug/carrier particles having elongation ratio greater than 1.6 for improved delivery by inhalation. Such particles are either produced by SCF technique or by a complex precipitation process. Electrospinning provides a direct, scalable process for the production of nanoparticles having greater elongation ratios.

U.S. Pat. No. 5,985,309 discloses large porous biodegradable microspheres containing proteins and peptides for pulmonary delivery.

It would be desirable to design a simple pharmaceutical composition which provides for all the positive attributes of the above dosage forms by combining the enhanced bioavailability of nanoparticles and physico-chemical characteristics of a nanofiber in a polymeric carrier matrix, in which the drug nanoparticles are homogeneously embedded, such that a convenient dosage form such as a rapid dissolve, immediate, delayed, modified release could be produced by simply selecting the appropriate polymer, without having to change the process.

### SUMMARY OF THE INVENTION

One object of the present invention is a process for electrospinning a pharmaceutically acceptable active agent, or agents, in the presence of a high molecular weight polymeric carrier that acts as viscosity enhancer and fiber forming agent. The process of making the electrospun pharmaceutical composition may be from a solution or a melt.

The present invention is also directed to a pharmaceutical composition comprising an electrospun fiber of a pharmaceutically acceptable polymeric carrier integrated with a pharmaceutically acceptable active agent.

The present invention is also directed to use of an electrospun pharmaceutical composition comprising a pharmaceutically acceptable active agent, and a pharmaceutically acceptable polymeric carrier directly for oral administration, pulmonary administration, or for dissolution into a liquid media for administration, such as a suspension or solution or by parenteral/intramuscular or intracavernosum injection.

### BRIEF DESCRIPTION OF THE DRAWINGS

FIG. 1 demonstrates electrospinning of viscous drug/polymer compositions either in solution or in melt form to produce nanofibers.

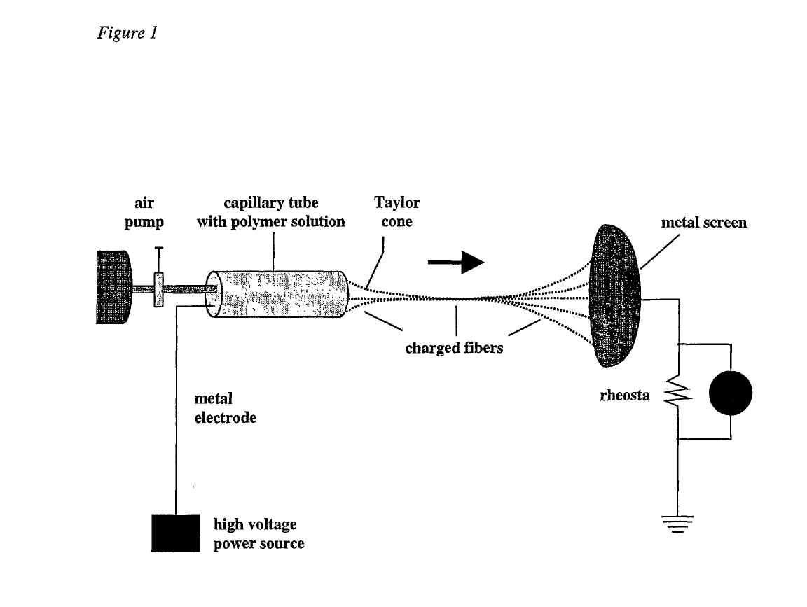

FIG. 2 shows the dissolution rate of nanofibers containing nabumetone normalized with respect to nanoparticles of nabumetone.

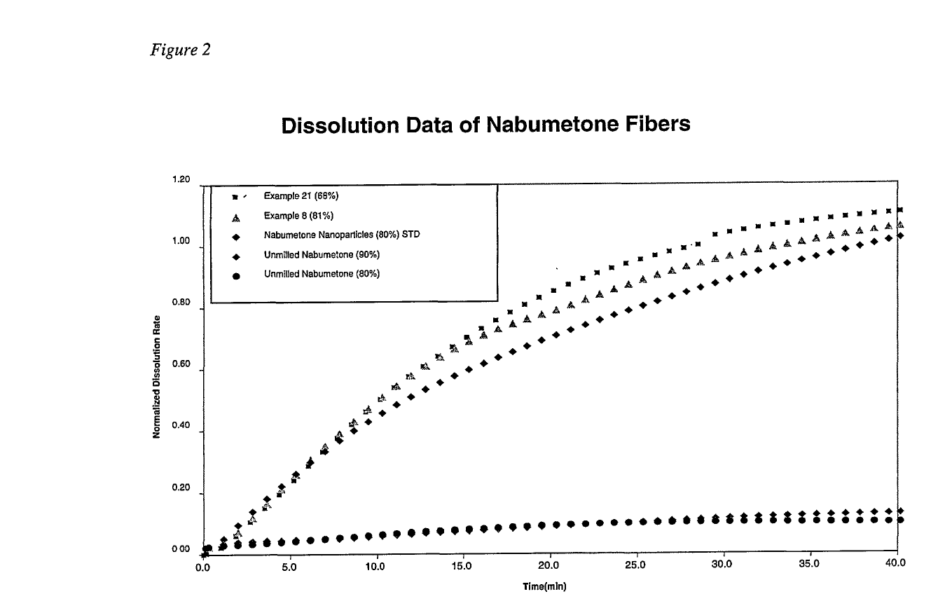

FIG. 3 shows a scanning electron microscope (SEM) of 60% w/w nabumetone spun with POLYOX® fibers.

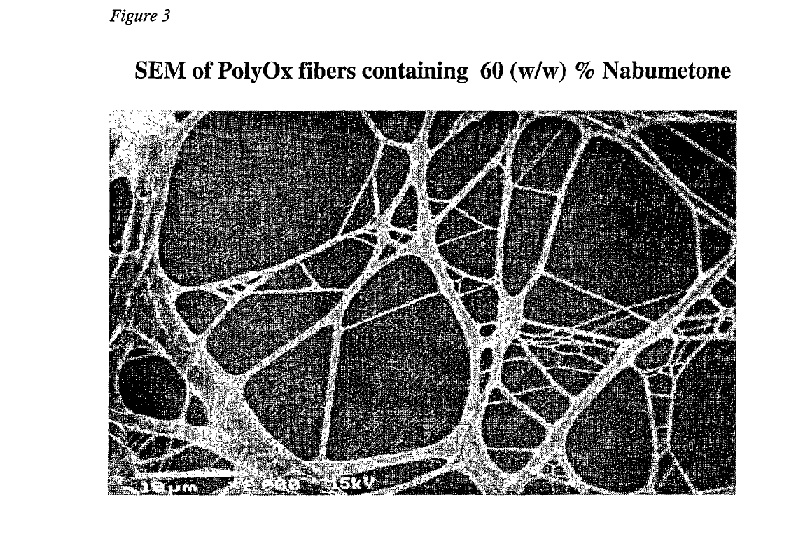

### DETAILED DESCRIPTION OF THE INVENTION

The present invention is directed to a novel composition of an electrospun fiber which fiber is the result of a high molecular weight polymeric carrier that acts as viscosity enhancer and fiber forming agent, and which carrier is spun with a pharmaceutically acceptable agent or drug.

As used herein the term “integrated” means that the drug is integrated with, admixed with, comingled with, or intermixed with the carrier. It is not coated on the surface of an electrospun fiber (woven or non-woven). Specifically the fiber contains both the agent and the carrier together, preferably in a homogeneous manner. While it is recognized that incomplete stirring of the solutions or the neat/melted compositions may result in some heterogenicity of the resultant fiber, the premise is that the drug and the carrier are spun together, rather than being applied in a later step to a fiber.

The electrospun fibers of the present invention are expected to have diameters in the nanometer range, and hence provide a very large surface area. The process generates fibers where a high surface to volume ratio is important. This extremely high surface area has profound influence on the bioavailability of a poorly water soluble drug, since it is known that increased surface can lead to increased dissolution rate.

A suitable dosage form, such as oral or parenteral form, including pulmonary administration, may be designed by judicious consideration of polymeric carriers, in terms of their physico-chemical properties as well as their regulatory status. Other pharmaceutically acceptable excipients may be included to ameliorate the stabilization or de-agglomeration of the drug nanoparticles. The pharmaceutical excipients might also have other attributes, such as absorption enhancers.

Electrospun pharmaceutical dosage form may be designed to provide rapid dissolution, immediate, delayed, or modified dissolution, such as sustained and /or pulsatile release characteristics.

Taste masking of the active agent can also be achieved by using polymers having functional groups capable of promoting specific interactions with the drug moiety. The electrospun dosage forms may be presented as compressed tablets, sachets or films. Conventional dosage forms such as immediate, delayed and modified release systems can be designed by appropriate choice of the polymeric carrier, drug combination, as described in the art.

It is one object of the present invention to provide pharmaceutically acceptable drug nanoparticles embedded homogeneously in polymeric nanofibers, such that the drug readily bioavailable independent of the route of administration.

Electrospinning, commonly referred to as electrostatic spinning, is a process of producing fibers, with diameters in the range of 100 nm. The process consists of applying a high voltage to a polymer solution or melt to produce a polymer jet. As the jet travels in air, the jet is elongated under repulsive electrostatic force to produce nanofibers. The process has been described in the literature since the 1930. A variety of polymers both natural and synthetic having optimal characteristics have been elctrospun under appropriate conditions to produce nanofibers, (see Reneker et al., Nanotechnology, 1996, 7, 216). Different applications have been suggested for these electrospun nanofibers, such as air filters, molecular composites, vascular grafts, and wound dressings.

U.S. Pat. No. 4,043,331, is intended for use as a wound dressing whereas U.S. Pat. No. 4,044,404, and U.S. Pat. No. 4,878,908 are tailored towards creating a blood compatible lining for a prosthetic device. All of the disclosed water insoluble polymers are not pharmaceutically acceptable for use herein, however the water soluble polymers disclosed are believed to be pharmaceutically acceptable. None of the preparations in these patents disclose a working example of an electrospun fiber with an active agent. The patents claim the use of enzymes, drugs and/or active carbon on the surface of the nanofibers, prepared by immobilizing the active moieties so that they act at the site of application and “do not percolate throughout the body”.

EP 542514, U.S. Pat. No. 5,311,884 and U.S. Pat. No. 5,522,879 pertain to use of spun fibers for a piezoelectric biomedical device. The piezoelectric properties of fluorinated polymers, such as those derived from a copolymer of vinylidene fluoride and tetrafluoroethylene are not considered pharmaceutically acceptable polymers for use herein.

U.S. Pat. No. 5,024,671 uses the electrospun porous fibers as a vascular graft material which is filled with a drug in order to achieve a direct delivery of the drug to the suture site. The porous graft material is impregnated (not electrospun) with the drug and a biodegradable polymer is added to modulate the drug release. The vascular grafts are also made from non-pharmaceutically acceptable polymers, such as the polytetrafluorethylene or blends thereof.

U.S. Pat. No. 5,376,116, U.S. Pat. No. 5,575,818, U.S. Pat. No. 5,632,772, U.S. Pat. No. 5,639,278 and U.S. Pat. No. 5,724,004 describe one form or another of a prosthetic device having a coating or lining of an electrospun non-pharmaceutically acceptable polymer. The electrospun outer layer is post-treated with a drug such as disclosed in the '116 patent (for breast prosthesis). The other patents describe the same technology and polymers but apply the technique to other applications, such as endoluminal grafts or endovascular stents.

Consequently, the present invention is the first to produce a pharmaceutical composition of an active agent(s) and a pharmaceutically acceptable polymer as an electrospun fiber. The homogenous nature of this process produces a quantity of fibers which allow for nanoparticles of drugs to be dispersed throughout. The size of particle, and quality of dispersion provide for a high surface area of drug. One use of the increased surface area of drug is improved bioavailability in the case of a poorly water soluble drug. Other uses would be for decreased drug-drug or enzymatic interactions.

The present invention is therefore directed to use in any form of a nanofibrous drug either alone, or in combination with a pharmaceutically acceptable polymer (or combination thereof) for enhancing the bioavailability of a drug, preferably a poorly water soluble drug.

The present invention is also directed to a rapidly dissolving dosage form comprising an electrospun water soluble polymer in combination with an active agent, such that the rapid dissolving dosage form disintegrates in a rapid manner, over a short time period, in the mouth or other suitable body cavity. In the oral context this would produce small particulate matter, which could be ingested without needing water.

A rapid dissolve dosage form may include a drug which is either water soluble or water insoluble. A rapid onset of action is a not prerequisite for a rapid dissolve dosage form. For a bitter tasting drug it may be advantageous to have it in an insoluble form, either by its own solubility characteristics or by polymer coating. Therefore, the main attribute of a rapid dissolve dosage form is that the excipients rapidly disintegrate in the mouth, exposing the drug particles to be easily swallowed. This being the case, electrospun polymer (water-soluble) nanofibers may suitably be premixed with drug during spinning or post mixed during fabrication of the rapid dissolve dosage form.

While the application of this process may be of use for incorporation of a pharmaceutically acceptable drug for topical delivery, it is primarily oriented towards oral, intravenous, intramuscular, or inhalation usage.

Pharmaceutically acceptable agents, actives or drugs as used herein, is meant to include active agents having a pharmacological activity for use in a mammal, preferably a human. The pharmacological activity may be prophylactic or for treatment of a disease state. The usage is not meant to include agricultural or insecticide usage for application to plants or soil. Use of the electrospun fiber as a woven or non-woven fabric for direct application as a topical treatment in wound dressing or in clothing is also not an aspect of the present invention. However, use of the fibers in a pharmaceutical formulation for topical administration are considered within the scope of the present invention.

As used herein the term's “active agent”, “drug moiety” or “drug” are used interchangeably.

Water solubility of the active agent is defined by the United States Pharmacoepia. Therefore, active agents which meet the criteria of very soluble, freely soluble, soluble and sparingly soluble as defined therein are encompassed this invention. It is believed that the electrospun polymeric composition which most benefit those drugs which are insoluble or sparingly soluble.

Suitable drug substances can be selected from a variety of known classes of drugs including, for example, analgesics, anti-inflammatory agents, anthelmintics, anti-arrhythmic agents, antibiotics (including penicillin's), anticoagulants, antidepressants, antidiabetic agents, antiepileptics, antihistamines, antihypertensive agents, antimuscarinic agents, antimycobactefial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, PDE IV inhibitors, NK3 inhibitors, CSBP/RK/p38 inhibitors, antipsychotics, vasodilators and xanthines.

Preferred drug substances include those intended for oral administration and intravenous administration. A description of these classes of drugs and a listing of species within each class can be found in Martindale, The Extra Pharmacopoeia, Twenty-ninth Edition, The Pharmaceutical Press, London, 1989, the disclosure of which is hereby incorporated herein by reference in its entirety. The drug substances are commercially available and/or can be prepared by techniques known in the art.

As noted, the electrospun composition may also be able to taste mask the many bitter or unpleasant tasting drugs, regardless of their solubility. Suitable active ingredients for incorporation into fibers of the present invention include the many bitter or unpleasant tasting drugs including but not limited to the histamine H₂-antagonists, such as, cimetidine, ranitidine, famotidine, nizatidine, etinidine; lupitidine, nifenidine, niperotidine, roxatidine, sulfotidine, tuvatidine and zaltidine; antibiotics, such as penicillin, ampicillin, amoxycillin, and erythromycin; acetaminophen; aspirin; caffeine, dextromethorphan, diphenhydramine, bromopheniramine, chloropheniramine, theophylline, spironolactone, NSAIDS's such as ibuprofen, ketoprofen, naprosyn, and nabumetone; 5HT₃ inhibitors, such as granisetron (Kytril®), or ondansetron (Zofran®); seratonin re-uptake inhibitors, such as paroxetine, fluoxetine, fluvoxamine, and sertraline; vitamins such as ascorbic acid, vitamin A, and vitamin D; dietary minerals and nutrients, such as calcium carbonate, calcium lactate, etc., or combinations thereof.

Suitably, the above noted active agents, in particular the anti-inflammatory agents, may also be combined with other active therapeutic agents, such as various steroids, decongestants, antihistamines, etc., as may be appropriate.

Preferably, the active agent is nabumetone, cis-4-Cyano-4-[3-cyclopentyloxyl)-4-methoxyphenyl]cyclohexanecarboxylic acid, ASA, paroxetine (Seroxat®), Ariflo, ropirinole (Requip®), rosiglitazone (Avandia®), or hydrochlorothyazie and traimeterene (Dyazide®).

Other suitable active agents are amprenavir (Agenerase®), lamivudine (Epivir®), epoprostenol (Flolan®), zanamivir (Relenza®), alosetron (Lotronex®), alelometasone (Aclovate®), beclomethasone (Beclovent® and Beconase®), malphalan (Aleran®), naratriptan (Amerge®), succinylcholine, cefuroxiime (Ceftin®), ceftazidime (Ceptaz®), cefuroxime (Zinacef®), zidovudine (Retrovir®), fluticasone (Flonase® or Cutivate®), pyrimethamine (Daraprim®), colfosceril, sumatriptan (Imitrex®), lamotrigine (Lamictal®), chlorambucil (Leukeran®), atovaquone (Malaron® or Mepron®), mivacurium (Mivacron®), busulfan (Myleran®), vinorelbine (Navelbine®), cisatracurium (Nimbex®), doxacurium (Nuromax®), atacurium (Tracrium®), oxiconazole (Oxistat®), mercaptopurine (Purinethol®) and thioguanine (Tabloid®), grepafloxacin (Raxar®), salmeterol (Serevent®), clobetasol (Temovate®), ranitidine, famotidine, omeprazole (R and S isomers), remifentanil (Ultiva®), valacyclovir (Valtrex®), acyclovir (Zovirax®), famciclovir (Famvir®), penciclovir (Denavir®), albuterol (Ventolin®), bupropion (Wellbutrin® or Zyban®), or abacavir (Ziagen®), 4-(3,4-dihydro-1-methyl-2(1H)-isoquinolinyl)-N-(4-fluorophenyl)-5,6-dimethyl-2-pyrimidinamine, (N-(2,6-dichlorobenzoyl)-4-(2,6-dimethoxyphenyl)-L-phenylalanine); telmisartan, lacidipine, eniluracil, amoxicillin (Amoxcil®), clavulanate, mupirocin, ticarcillin, cerivastatin (Baycol®), carvedilol (Coreg®), topotecan (Hycamtin®)), Factive®, Locilex®, Novastan®, Tranilast, Lotrifiban, 8-[(4-Amino-1-methylbutyl)amino]-2,6-dimethoxy-4-methyl-5-(3-trifluoromethylphenoxy)quinoline succinate, (1S,2R,3S)-1-(1,3-Benzodioxol-5-yl)-2,3-dihydro-3-[2-(2-hydroxyethoxy)-4-methoxyphenyl]-5-propoxy-1H-indene-2-carboxylic acid, nelarabine, dutasteride, maribavir, 3-(3-{1-[(Isopropyl-phenyl-carbamoyl)-methyl]-2,4-dioxo-5-phenyl -2,3,4,5-tetrahydro-1H-benzo[b][1,4]diazepin-3-yl}-ureido)-benzoic acid; 6-amino -3-(2,3,5-trichlorophenyl)pyrazin-2-ylamine; (2R,3R,4S,5R)-2-[6-Amino-2-(1S-hydroxymethyl -2-phenyl-ethylamino)-purin-9-yl]-5-(2-ethyl-2H-tetrazol-5-yl)-tetrahydro-furan -3,4-diol; (6α,11β,16α,17α)-6,9-difluoro-11-hydroxy-16-methyl-3-oxo-17-({[(3S)-2-oxotetrahydrofuran-3-yl]thio}carbonyl)androsta-1,4-dien-17-yl propionate; (3S)-tetrahydrofuran-3-yl (1S,2R)-3-[[(4-aminophenyl)sulfonyl](isobutyl)amino]-1-benzyl-2-(phosphonooxy)propylcarbamate; (3R,5R)-3-Butyl-3-ethyl-7,8-dimethoxy-5-phenyl-2,3,4,5-tetrahydro-1,4-benzothiazepine 1,1-dioxide; (1S,3S,4S,8R)-3-(3,4-dichlorophenyl)-7-azatricyclo[5.3.0.0^{4,8}]decan-5-ol; (2S,3S,5R)-2-(3,5-Difluorophenyl)-3,5-dimethyl-2-morpholinol; (S)-2-(2-Benzoyl-phenylamino) -3-{4-[2-(5-methyl-2-phenyl-oxazol-4-yl)-ethoxy]-phenyl}-propionic acid; 3′-[(2-{[(2R)-2-(3-chlorophenyl)-2-hydroxyethyl]amino}ethyl)amino] [1,1′-biphenyl]-3-carboxylic acid; (2S)-2-{[(1Z)-1-methyl-3-oxo-3-phenylprop-1-enyl]amino}-3-{4-[2-(5-methyl-2-phenyl-1,3-oxazol-4-yl)ethoxy]phenyl}propanoic acid; or combinations and mixtures thereof of all compounds noted herein.

In short, for use herein, a poorly soluble drug should have good solubility in an organic solvent, or a poorly soluble drug must be useable in a melt process as further described below.

The nanofibers of this invention will contain high molecular weight polymeric carriers. These polymers, by virtue of their high molecular weight, form viscous solutions which can produce nanofibers, when subjected to an electrostatic potential.

Suitable polymeric carriers can be preferably selected from known pharmaceutical excipients. The physico-chemical characteristics of these polymers dictate the design of the dosage form, such as rapid dissolve, immediate release, delayed release, modified release such as sustained release, or controlled release, pulsatile release etc.

DNA fibers have been used to form fibers by electrospinning, Fang et al., J. Macromol. Sci.-Phys., B36(2), 169-173 (1997). Incorporation of a pharmaceutically acceptable active agent, such as a biological agent, a vaccine, or a peptide, with DNA, RNA or derivatives thereof as a spun fiber is also within the scope of this invention.

The fiber forming characteristics of the polymer are exploited in the fabrication of nanofibers. Hence, molecular weight of the polymer is the single most important parameter for choice of polymer. As previously noted, a large number of polymers have already been electrospun, such as cellulose acetate, PVA, PEO, PVP, polyacrylamide, polyurethane, polycarbonate, PTFE, PE, PP, polyacrylate, Kevlar, PHB, polyaniline, DNA, poly (phenylene terphthalamide) and silk.

However, for purposes herein additional representative examples of polymers suitable for pharmaceutical applications, include, but are not limited to, poly(ethylene oxide), polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives such as the Eudragit family of polymers available from Rohm Pharma, poly(alpha-hydroxy acids) and its copolymers such poly(caprolactone), poly(lactide-co-glycolide), poly(alpha-aminoacids) and its copolymers, poly(orthoesters), polyphosphazenes, poly(phosphoesters), and polyanhydrides, or mixtures thereof.

Most of these pharmaceutically acceptable polymers are described in detail in the Handbook of Pharmaceutical excipients, published jointly by the American Pharmaceutical association and the Pharmaceutical society of Britain.

Preferably, the polymeric carriers are divided into three categories: (1)water soluble polymers useful for rapid dissolve and immediate release of active agents, (2) water insoluble polymers useful for controlled release of the active agents; and (3) pH sensitive polymers for pulsatile or targeted release of active agents. It is recognized that combinations of both carriers may be used herein. It is also recognized that several of the polyacrylates are pH dependent for the solubility and may fall into both categories.

Water soluble polymers include but are not limited to, poly(ethylene oxide), polyvinyl alcohol, polyvinyl pyrrolidone, hyaluronic acid, alginates, carragenen, cellulose derivatives such as carboxymethyl cellulose sodium, hydroxyethyl cellulose, hydroxypropylcellulose, hydroxypropylmethyl cellulose, hydroxypropylmethyl cellulose phthalate, cellulose acetate phthalate, starch and its derivatives such as hydroxyethyl starch, sodium starch glycolate, dextrin, chitosan and its derivatives, albumen, zein, gelatin, and collagen.

Preferably, a water soluble polymer for use herein is polyethylene oxide, such as the brand name POLYOX®. It is recognized that the polymers may be used in varying molecular weights, with combinations of molecular weights for one polymer being used, such as 100K, 200K, 300K, 400K, 900K and 2000K. Sentry POLYOX is a water soluble resin which is listed in the NF and have approximate molecular weights from 100K to 900K and 1000K to 7000K. These commercially available polymers may be used as 1%, 2% and 5% solutions (depending upon molecular weight).

NF grades of Sentry POLYOX, a water soluble resin is available with varying molecular weights as noted above. A table, shown below, provides further information on the grade vs. approx. molecular weight for use in the examples herein.

| | Approximate | Viscosity range at 25° C., cP | | |
|---|---|---|---|---|
| NF Grade | mol. weight | 5% solution | 2% solution | 1% solution |
| WSRN-10 | 100,000 | 30-50 | | |
| WSRN-80L | 200,000 | 500 | | |
| WSRN-80H | 200,000 | 90-105 | | |
| WSRN-750 | 300,000 | 500-1200 | | |
| WSRN-3000 | 400,000 | 1,250-4,500 | | |
| WSR-20S | 600,000 | 4,500-8,800 | | |
| WSR-1105 | 900,000 | 8,800-17,600 | | |
| WSRN-12K | 1,000,000 | | 400-800 | |
| WSRN-60K | 2,000,000 | | 2,000-4,000 | |
| WSR-301 | 4,000,000 | | | 1,500-4,500 |
| WSR | 5,000,000 | | | 4,500-7,500 |
| coagulant | | | | |
| WSR-303 | 7,000,000 | | | 7,500- |
| | | | | 10,000 |

Additional preferred polymers include povidone, having K values and molecular weight ranges from:

| K value | Mol. wt. |
|---|---|
| 12 | 25 |
| 15 | 8000 |
| 17 | 10,000 |
| 25 | 30,000 |
| 30 | 50,000 |
| 60 | 400K |
| 90 | 1000K |
| 120 | 3000K |

Water insoluble polymers include but are not limited to, polyvinyl acetate, methyl cellulose, ethylcellulose, noncrystalline cellulose, polyacrylates and its derivatives such as the Eudragit family of polymers available from Rohm Pharma (Germany), poly(alpha-hydroxy acids) and its copolymers such as poly(ε-caprolactone), poly(lactide-co-glycolide), poly(alpha-aminoacids) and its copolymers, poly(orthoesters), polyphosphazenes, poly(phosphoesters), and polyanhydrides.

These pharmaceutically acceptable polymers and their derivatives are commercially available and/or be prepared by techniques known in the art. By derivatives it is meant, polymers of varying molecular weight, modification of functional groups of the polymers, or co-polymers of these agents, or mixtures thereof.

Further, two or more polymers can be used in combination to form the fibers as noted herein. Such combination may enhance fiber formation or achieve a desired drug release profile.

The choice of polymers taken with the active agent may provide suitable taste masking functions for the active agents. For instance, use of an ionic polymer of contrasting charge, such as a cationic polymer complexed with an anionic active agent, or an anionic polymer complexed with a cationic active agent may produce the desired results. Addition of a second taste masking agent, such as a suitable cyclodextrin, or its derivatives may also be used herein.

The polymeric composition may be electrospun from a solvent base or neat (as a melt). Solvent choice is preferably based upon the solubility of the active agent. Suitably, water is the best solvent for a water soluble active agent, and a water soluble polymer like POLYOX. Alternatively, water and a water-miscible organic solvent may used. However, it is necessary to use an organic solvent to prepare a homogenous solution of the drug with polymer when the drug is non-water soluble, or sparingly soluble.

It is recognized that these polymeric composition which are spun neat may also contain additional additives such as, plasticizers. The plasticizers are employed to assist in the melting characteristics of the composition. Exemplary of plasticizers that may be employed in this invention are triethyl citrate, triacetin, tributyl citrate, acetyl triethyl citrate, acetyl tributyl citrate, dibutyl phthalate, dibutyl sebacate, vinyl pyrrolidone, propylene glycol, glycol tiracetate, polyethylene glycol, or polyoxyethylene sorbitan monolaurate and combinations or mixtures thereof.

Preferably, the solvent of choice is a GRASS approved organic solvent, although the solvent may not necessarily be “pharmaceutically acceptable” one, as the resulting amounts may fall below detectable, or set limits for human consumption they may be used. It is suggested that ICH guidelines be used for selection. GRASS in an anacronym for “generally recognized as safe”.

Suitable solvents for use herein include, but are not limited to acetic acid, acetone, acetonitrile, methanol, ethanol, propanol, ethyl acetate, propyl acetate, butyl acetate, butanol, N,N dimethyl acetamide, N,N dimethyl formamide, 1-methyl-2-pyrrolidone, dimethyl sulfoxide, diethyl ether, disisopropyl ether, tetrahydrofuran, pentane, hexane, 2-methoxyethanol, formamide, formic acid, hexane, heptane, ethylene glycol, dioxane, 2-ethoxyethanol, trifluoroacetic acid, methyl isopropyl ketone, methyl ethyl ketone, dimethoxy propane, methylene chloride etc., or mixtures thereof.

A preferred solvent is a mixture of water and acetonitrile, or water and acetone.

The solvent to polymeric composition ratio is suitable determined by the desired viscosity of the resulting formulation.

For electrospinning of a pharmaceutical polymeric composition, key parameters are viscosity, surface tension, and electrical conductivity of the solvent/polymeric composition.

By the term “nanoparticulate drug” as used herein, is meant, nanoparticule size of an active agent within the electrospun fiber.

The polymeric carriers may also act as surface modifiers for the nanoparticulate drug. However, a second oligomeric surface modifier may also be added to the electrospinning solution. All of these surface modifiers may physically adsorb to the surface of the drug nanoparticles, so as to prevent them agglomerating.

Representative examples of these second oligomeric surface modifier or excipients, include but are not limited to: Pluronics® (block copolymers of ethylene oxide and propylene oxide), lecithin, Aerosol OT™ (sodium dioctyl sulfosuccinate), sodium lauryl sulfate, polyoxyethylene sorbitan fatty acid esters, i.e., the polysorbates such as Tween™, such as Tween 20, 60 & 80, the sorbitan fatty acid esters, i.e., sorbitan monolaurate, monooleate, monopalmitate, monosterate, etc. such as Span™ or Arlacel™, Emsorb™, Capmul™, or Sorbester™, Triton X-200, polyethylene glycols, glyceryl monostearate, Vitamin E-TPGS™ (d-alpha-tocopheryl polyethylene glycol 1000 succinate), sucrose fatty acid esters, such as sucrose stearate, sucrose oleate, sucrose palmitate, sucrose laurate, and sucrose acetate butyrate, etc.

Surfactants are added on a weight/weight basis to the drug composition. Suitably, the surfactants are added in amounts of about 10%, preferably about 5% or less. Surfactants can lower the viscosity and surface tension of the formulation, and in higher amounts can adversely effect the quality of the electrospun fibers.

The surfactant selection may be guided by HLB values but is not necessarily a useful criteria. While HLB surfactants have been utilized herein, such as Tween™ 80 (HLB=10), Pluronic F68 (HLB=28), and SDS (HLB>40), lower HLB value surfactants, such as Pluronic F92 may also be used.

Another pharmaceutically acceptable excipients may be added to the electrospinning composition. These excipients may be generally classified as absorption enhancers, additional surfactants, flavouring agents, dyes, etc.

Suitable flavoring agents for use herein include, but are not limited to, wintergreen; orange, grapefruit, and cherry-raspberry; While w/w % will vary for each composition, the flavouring agent should be present from about 0.25 to about 5% w/w of the total formulation.

Suitable coloring agents, pigments, or dyes, such as FD&C or D&C approved lakes and dyes, iron oxide and titanium dioxide may also be included in the formulations. The amount of pigment present may be from about 0.1% to about 2.0% by weight of the composition.

Additionally, the formulation may also contain sweeteners such as various natural sugars, aspartame, sodium cyclamate and sodium saccharinate; as well as the flavorants such as those noted above.

The polymeric carriers or the second oligomeric surface modifiers, if appropriately chosen, may themselves act as absorption enhancers, depending on the drug. Suitable absorption enhancers for use herein, include but are not limited to, chitosan, lecithin, lectins, sucrose fatty acid esters such as the ones derived from stearic acid, oleic acid, palmitic acid, lauric acid, and Vitamin E-TPGS.

Use of the electrospun composition herein may be by conventional capsule or tablet fill. Alternatively, the fibers may be ground, suitably by cryogenic means, for compression into a tablet or capsule, for use by inhalation, or parenteral administration. The fibers may also be dispersed into an aqueous solution which may then be directly administered by inhaled or given orally. The fibers may also be cut and processed as a sheet for further administration with agents to form a polymeric film, which may be quick-dissolving.

Another aspect of the present invention is an alternative electrospinning process for making the pharmaceutical compositions described herein. The working examples herein electrostatically charge the solution whereas the pharmaceutical composition may also be ejected from a sprayer onto a receiving surface which is electrostatically charged and placed at an appropriate distance from the sprayer. As the ejectant travels in the air from the sprayer towards the charged collector, fibers are formed. The collectors can be either a metal screen, or in the form of a moving belt. The fibers may be deposited on a moving belt which could be continuously removed and taken away for further processing as desired.

In a preferred embodiment of the invention for water insoluble agents, is the active nabumetone, electrospun in w/w % ranges from 0 to 82%, with 200K, 400K, 900K and 2000K POLYOX, and Tween 80, SDS, Pluronic F68, or TPGS. A preferred solvent system is water/acetonitrile.

### EXAMPLES

The invention will now be described by reference to the following examples which are merely illustrative and are not to be construed as a limitation of the scope of the present invention. All temperatures are given in degrees centigrade, all solvents are highest available purity unless otherwise indicated.

### Example 1

### Electrospinning of 25% (w/w) Aspirin Composition

A stock solution of 2.5% solution of POLYOX WSR N-60K™ (Union Carbide) was prepared in MilliQ™ water by gentle mixing in a shaking water bath. 10 milliliters (hereinafter “mL” or “ml”) of this POLYOX solution was added to a solution of 0.12 grams (hereinafter “g”) Acetylsalicylic acid (Sigma) in 0.5 mL acetone. The contents were thoroughly mixed and 1 mL of acetone was added to obtain a clear solution. This solution was transferred to a 25 mL glass vessel having a 0.03millimeter (hereinafter “mm”) capillary outlet at the bottom and two inlets, one for applying a positive Helium (He) pressure and the other for introducing the electrode. The electrode was connected to the positive terminal of a high voltage power supply (ModelD-ES30P/M692, Gamma High Voltage Research Inc. FL). The ground from the high voltage power supply was connected to a rotating drum covered with aluminum foil. The inlet Helium pressure was at 2.5 psi and a voltage of +14.5 KV was applied to the solution. The dry fibers were collected on a drum rotating a speed of 50-60 rpm. The fibers were peeled off the drum.

The electrospinning process is further described in J. Doshi's Dissertation, of “The ElectroSpinning Process and Applications of Electrospun Fibers”, August 1994, University of Akron, which is incorporated herein by reference in its entirety.

### Example 2

### Electrospinning-of 25% Nabumetone Composition

A stock solution of 30% Polyethylene oxide (Molecular weight 400K, Aldrich) was prepared in MilliQ™ water by gentle shaking. 5 mL of this 30% solution was added to 0.5 g nabumetone (SB Corporation) dissolved in 6 ml of acetonitrile. The contents were gently stirred and another 5 mL acetonitrile was added in small portions until a clear solution was obtained. 0.1 ml of Tween™ 80 (Sigma) was added to the solution. This solution was electrospun using the same conditions described above in Example 1. Fibers were collected and removed from the drum.

### Example 3

### Electrospinning of 30% Nabumetone Composition

A stock solution of 7.5% (w/v) POLYOX® WSR N-3000 (Molecular weight of approx. 400K, Union Carbide) in MilliQ™ water/acetonitrile was prepared by mixing 15 g of PEO in 50 ml water and 150 mL acetonitrile.

To 10 mL of this solution was added 0.4 g nabumetone along with acetonitrile and 0.2 mL Tween™ 80 to obtain a homogeneous solution. This solution was electrospun under the same conditions as described above in example 1 to yield 1.3 g fibers.

### Example 4

### Electrospinning of 50% Nabumetone Composition

To 10 mL of the stock solution of water/acetonitrile from Example 3 above was added 0.8 g nabumetone. The solution was homogenized by adding 1 mL acetonitrile along with 0.2 mL Tween™ 80. The solution was spun using similar conditions to Example 1 above, but using a feed pressure of 2 psi and 16 KV to yield 1.2 g of fibers.

### Example 5

### Electrospinning of 70% Nabumetone Composition

To 5 mL of the POLYOX® N-3000 solution from Example 3 was added 0.86 g of nabumetone. The solution was made homogeneous by adding 1.6 mL of acetonitrile along with 0.1 mL Tween™ 80. The solution was spun using similar conditions to Example 1 above, but using a feed pressure of 0.5 psi and 16 KV to yield 0.93 g of fibers.

### Example 6

### Electrospinning of 80% Nabumetone Composition

To a mixture of 2 g Nabumetone, 0.1 g SDS (J T Baker) and 0.4 g POLYOX®WSR-1105 (900K) was added 1.2 mL MilliQ® water and 10.5 mL acetonitrile. This mixture was left in a shaking water bath at 37° C. until all solid material dissolves to form a viscous solution. The resultant solution was electrospun using conditions similar to Example 1 above, but using a feed pressure of 2 psi and 18 KV to yield 2.1 g of fibers.

### Example 7

### Electrospinning of 80% Nabumetone Composition

To a mixture of 2 g Nabumetone, 0.05 g Pluronic® F68 (BASF) and 0.4 g POLYOX® WSR-1105 (900K) was added 1 mL MilliQ® water and 12 mL acetonitrile. This mixture was left in a shaking water bath at 37° C. until all solid material dissolves to form a viscous solution. The resultant solution was electrospun using conditions similar to Example 1 above, but using a feed pressure of 2 psi and 18 KV to yield 2.1 g of fibers.

### Example 8

### Electrospinning of 80% Nabumetone Composition

Two grams of Nabumetone was dissolved in 11 mL of acetonitrile. To the solution was added 0.1 g of Vitamin E-TPGS (Eastman) and 0.4 g POLYOX® WSR-1105 (900K). The mixture was left in a shaking water bath at 37° C. until all solid material dissolves to form a viscous solution. The resultant solution was electrospun using conditions similar to Example 1 above, but using a feed pressure of 0.5 psi and 16 KV to yield 2 g of fibers.

### Example 9

### Determination of Nabumetone Content in the Nanofiber Composition

Accurately weighed out 20 to 50 mg (depending on the expected drug content) of a nanofiber composition, such as described above, into a scintillation vial and dissolved it 5 mL acetonitrile/water (80/20) mixture. The solution was quantitatively transferred to a 50 mL volumetric flask using acetonitrile/water (80/20) and made up to volume (50 mL) using acetonitrile/water as diluent. Three different samples taken from different parts of fibrous sheets were prepared to determine the macroscopic heterogeneity within the fibers.

A standard solution of nabumetone was prepared using accurately weighed sample of 20 mg nabumetone in a 100 mL volumetric flask. The sample was made up using acetonitrile/water(80:20) as a diluent. 20 uL of this solution was injected in Waters HPLC system equipped with Waters 550 pumps, 717plus autosampler, and Spectroflow 783 UV detector. The data acquisition was carried out through a PE Nelson Box and Turbochrom (PE) software. The mobile phase consisted of acetonitrile/water/acetic acid in the volume ratio of 44/55/1. The flow rate was 1.4 ml/min and the detection was done at 254 nm.

| | Nabumetone Content (wt. %) | | |
|---|---|---|---|
| | Sample #1 | sample #2 | Sample #3 |
| Example 8 | 81.2 | 79.5 | 81.2 |
| Example 6 | 82.9 | 82.8 | 83.0 |
| Example 5 | 59 | 61.2 | 60.8 |
| Example 4 | 36 | 36.9 | 35 |
| Example 3 | 30 | 30.5 | 29.8 |

### Example 10

### Residual Solvent Analysis in the Nabumetone Nanofibers

Residual solvent analysis was carried out at QTI (Whitehouse, N.J.) using samples dissolved in DMSO (dimethyl sulfoxide) and quantitated by capillary Gas Chromatography. The results, shown in the Table below demonstrate that all the samples analyzed contained less 100 ppm of acetonitrile.

| | TABLE |
|---|---|
| | acetonitrile content |
| Example 5 | <100 ppm |
| Example 4 | <100 ppm |
| Example 3 | <100 ppm |

### Example 11

### In-Vitro Dissolution Assay

The equipment used for this procedure is a modified USP4, the major differences being: 1) low volume cell; 2)stirred cell; 3) retaining filters which are adequate at retaining sub micron material. The total run time is 20 minutes. with 2.5 mg of drug (weigh proportionally more formulated material).

Flow Cell Description: Swinnex filter assemblies obtained from Millipore, having 0.2 micron Cellulose Nitrate membranes. (Millipore, Mass.) as internal filters. The internal volume of the cell is approximately 2 mL. A Small PTE stirrer customized to fit the Swinnex assembly (Radleys Lab Equipment Halfround Spinvane F37136) is used. The dissolution medium is water at a flow rate of 5 mL/min. The whole set up is placed at a thermostat of 37° C. The drug concentration is measured by passing the elutent through a UV detector having a flow cell dimension of 10 mm. The UV detection is carried out at 284 nm.

Determination of Extent of Drug Solubility

The experimentation is designed to evaluate drug dissolution rate. As such it is unlikely with poorly soluble drugs and with water as the dissolution medium that 100% of the drug will dissolve in the 20 minute duration of the test. To determine the extent of drug solubility over this period collect all 100 ml of solution that elutes from the dissolution cell. Using a conventional UV spectrophotometer compare this solution against a reference solution of 2.5 mg of active agent, for instance Nabumetone, dissolved in 50/50 methanol/water. (For Nabumetone this can be prepared by 10 fold dilution of a solution containing 25 mg Nabumetone in 100 mls of 50/50 methanol/water). A suitable wavelength for comparison is 260 nm.

### Example 12

### Determination of Thermal Behavior of Nabumetone Containing Nanofibers.

Thermal studies on nabumetone nanofibers were performed on a MDSC TA (Wilmington, Del.). The samples were heated from 0 to 120° C. at 2° C./min at a modulation frequency of ±0.159° C. every 30 seconds. The nanofibers containing nabumetone two distinct endotherms at 50° C. and 75° C. corresponding to the melting of POLYOX and nabumetone respectively, when the nabumetone content is above 30% (wt.), below which only one melting endotherm is visible either due to the formation of a eutectic mixture or because the endotherms overlap.

| | Nabumetone | Melting of | Melting of |
|---|---|---|---|
| | content | POLYOX | Nabumetone |
| | (wt. %) | ° C. and ΔH | ° C. and ΔH |
| Example 8 | 81.2 | 49.4 (22.2 J/g) | 75 (80 J/g) |
| Example 7 | 84.4 | 51.5 (22.4 J/g) | 75.3 (82.4 J/g) |
| Example 6 | 82.9 | 50.5 (19.6 J/g) | 75.3 (87.3 J/g) |
| Example 5 | 60.3 | 49.2 (87.4 J/g) | 69 (86.2 J/g) |
| Example 4 | 35.9 | 45.1 (69.1 J/g) | 59 (7.39 J/g) |
| Example 3 | 30.1 | 47 (101 J/g) | |
| Example 2 | 29.3 | 48 (94.5 J/g) | |

### Example 13

### Electrospinning of 40% cis-4-Cyano-4-[3-Cyclopentyloxyl)-4-Methoxyphenyl]Cyclohexanecarboxylic Acid Composition

To 10 ml of the POLYOX WSRN-3000 solution from Example 3 was added 0.5 g of cis-4-Cyano-4-[3-cyclopentyloxyl)-4-methoxyphenyl]cyclohexanecarboxylic acid along with 1 mL acetonitrile and 0.1 mL Tween™ 80 to obtain a homogeneous solution. This solution was electrospun under the same conditions as described above in Example 1 to yield nanofibers containing the title compound.

### Example 14

### Electrospinning of (S)-3-Hydroxy-2-Phenyl-N-(1-Phenylpropyl)-4-Quinolinecarboxamide Compositions

Four Hundred milligrams of (S)-3-Hydroxy-2-phenyl-N-(1-phenylpropyl)-4-quinolinecarboxamide was dissolved in 5 mL of tetrahydrofuran (G. T Baker). To this solution 450 mg of POLYOX® WSR-1105 (900K) and 50 mg Vitamin E-TPGS(Eastman) were added. The mixture was left in a shaking water bath at 37° C. until all solid material dissolves to form a viscous solution. The viscosity of the solution was reduced by added 5 mL of acetonitrile. The resultant solution was electrospun using conditions similar to Example 1 above, but using a feed pressure of 0.5 psi and 16 KV to yield 0.5 g of fibers.

### Example 15

### Electrospinning of 4-[2-(Dipropylamino)Ethyl]-1,3-Dihydro-2H-Indol-2-One Monohydrochloride

Two hundred milligrams of Ropirinole was dissolved in 15 ml of milliQ water. To this solution was added 1 g of POLYOX® WSR N3000 NF and 50 mg of Tween 80. The mixture was left shaking in a water bath at 37° C. until all solid material dissolved to form a clear viscous solution. This solution was electrospun using a conditions similar to Example 1, at a feed pressure of 1 psi and 16 KV to yield 0.8 g of the material.

### Example 16

### Electrospinning of 4-[2-(Dipropylamino)Ethyl]-1,3-Dihydro-2H-Indol-2-One Monohydrochloride

Three hundred Fifty milligrams of Ropirinole was dissolved in 15 ml of milliQ water. To this solution was added 650 mg of POLYOX® WSR N3000 NF and 50 mg of Tween 80. The mixture was left shaking in a water bath at 37° C. until all solid material dissolved to form a clear viscous solution. This solution was electrospun using a conditions similar to Example 1, at a feed pressure of 1 psi and 16 KV to yield 0.7 g of the material.

### Example 17

### Electrospinning of Paroxetine

One hundred milligrams of paroxetine was dissolved in 20 ml of milliQ water. To this solution was added 800 mg of POLYOX® WSR N3000 NF and 50 mg of Tween 80. The mixture was left shaking in a water bath at 37° C. until all solid material dissolved to form a clear viscous solution. This solution was electrospun using a conditions similar to Example 1, at a feed pressure of 1 psi and 16 KV to yield 0.75 g of the material.

### Example 18

### Electrospinning of Kytril

Three hundred milligrams of Kytril was dissolved in 15 ml of milliQ water. To this solution was added 650 mg of POLYOX® WSR N3000 NF and 50 mg of Tween 80. The mixture was left shaking in a water bath at 37° C. until all solid material dissolved to form a clear viscous solution. This solution was electrospun using a conditions similar to Example 1, at a feed pressure of 1 psi and 16 kV to yield 0.7 g of the material.

### Example 19

### Electrospinning of 10% 2,3-Dihydro-5-Methyl-N-[6-(2-Pyridinylmethoxy)-3-Pyridinyl]-6-(Trifluoromethyl)-1H-Indole-1-Carboxamide Composition

Eight Hundred and Fifty milligrams of POLYOX® WSR-1105 (900K) was dissolved in 20 ml acetonitrile by shaking overnight in a water bath at 35° C. This forms a thick viscous solution. 5 ml of n-methyl pyrrolidone (NMP) and 50 mg of Vitamin E-TPGS(Eastman) were added to the solution and stirred. 100 mg of the title compound dissolved in 1 ml of NMP was added to the polymer solution. The clear solution obtained was electrospun under identical conditions to Example 1, to yield 0.5 g of the product.

### Example 20

### Electrospinning of 20% 2,3-Dihydro-5-Methyl-N-[6-(2-Pyridinylmethoxy)-3-Pyridinyl]-6-(Trifluoromethyl)-1H-Indole-1-Carboxamide Composition

Seven Hundred and Fifty milligrams of POLYOX® WSR-1105 (900K) was dissolved in 20 ml acetonitrile by shaking overnight in a water bath at 35° C. This forms a thick viscous solution. 5 ml of n-methyl pyrrolidone (NMP) and 50 mg of Vitamin E-TPGS (Eastman) were added to the solution and stirred. 200 mg of the title compound dissolved in 1 ml of NMP was added to the polymer solution. The clear solution obtained was electrospun under identical conditions to Example 1, to yield 0.7 g of the product.

### Example 21

### Electrospinning of 68% Nabumetone Composition

Three grams of Nabumetone was dissolved in 20 mL of acetonitrile. To the solution was added 0.25 g of Vitamin E-TPGS(Eastman), 0.8 g POLYOX WSR-1105 (900K) and 0.25 g Tween 80. The mixture was left in a shaking water bath at 37° C. until all solid material dissolves to form a viscous solution. The resultant solution was electrospun using conditions similar to Example 1 above, but using a feed pressure of 0.5 psi and 16 KV to yield 3.5 g of fibers.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A pharmaceutical composition comprising an electrospun fiber of a pharmaceutically acceptable polymeric carrier integrated with a pharmaceutically acceptable active agent which agent is sparingly water soluble or water insoluble.

2. The composition according to claim 1 wherein the active agent is nanoparticle in size.

3. The composition according to claim 1 wherein the active agent is homogenously dispersed with the carrier in the fiber.

4. The composition according to claim 1 or 2 wherein the active agent is water insoluble.

5. The composition according to claim 1 wherein the active agent is sparingly water soluble.

6. The composition according to claim 1 or 2 wherein the polymeric carrier is water soluble.

7. The composition according to claim 1 or 2 wherein the polymeric carrier is water insoluble.

8. The composition according to claim 1 wherein the composition further comprises a surfactant which is a block copolymer of ethylene oxide and propylene oxide, lecithin, sodium dioctyl sulfosuccinate, sodium lauryl sulfate, Tween 20, 60 & 80, Span™, Arlacel™, TritonX-200, polyethylene glycol, glyceryl monostearate, d-alpha-tocopheryl polyethylene glycol 1000 succinate, sucrose fatty acid ester, such as sucrose stearate, sucrose oleate, sucrose palmitate, sucrose laurate, sucrose acetate butyrate, or a mixture thereof.

9. The composition according to claim 1 or 8 wherein the composition further comprises an absorption enhancer.

10. The composition according to claim 1 which provides a taste masking effect of the active agent.

11. The composition according to claim 6 wherein the polymeric carrier is poly(ethylene oxide), polyvinyl alcohol, polyvinyl pyrrolidone, hyaluronic acid, alginates, carragenen, carboxymethyl cellulose sodium, hydroxyethyl cellulose, hydroxypropylcellulose, hydroxypropylmethyl cellulose, hydroxypropylmethyl cellulose phthalate, cellulose acetate phthalate, starch, hydroxyethyl starch, sodium starch glycolate, chitosan and its derivatives, albumen, gelatin, or collagen.

12. The composition according to claim 1 wherein the polymeric carrier is polyvinyl acetate, methyl cellulose, ethylcellulose, noncrystalline cellulose, polyacrylates and its derivatives, poly(alpha-hydroxy acids) and its copolymers such poly(caprolactone), poly(lactide-co-glycolide), poly(alpha-aminoacids) and its copolymers, poly(orthoesters), polyphosphazenes, poly(phosphoesters), or polyanhydrides.

13. The composition according to claim 1 wherein said drug substance is an analgesic, anti-inflammatory agent, anthelmintic, anti-arrhythmic agent, an antibiotic, anticoagulant, antidepressant, antidiabetic agent, antiepileptic, antihistamine, antihypertensive agent, antimuscarinic agent, antimycobacterial agent, antineoplastic agent, immunosuppressant, antithyroid agent, antiviral agent, anxiolytic sedative, astringent, beta-adrenoceptor blocking agent, contrast media, corticosteroid, cough suppressant, diuretic, dopaminergic, homeostatic, immunological agent, lipid regulating agent, muscle relaxant, parasympathomimetic, parathyroid, calcitonin, prostaglandin, radio-pharmaceutical, sex hormone, steroid, anti-allergic agent, antihistaminic, stimulant, sympathomimetic, thyroid agent, vasodilator, PDE IV inhibitor, or a mixture thereof.

14. The composition according to claim 13 wherein the drug substance is an anti-inflammatory agent or a PDE IV inhibitor.

15. The composition according to claim 14 wherein the said active is nabumetone, aspirin, cis-4-Cyano-4-[3-cyclopentyloxyl)-4-methoxyphenyl]-cyclohexanecarboxylic acid, or (S)-3-Hydroxy-2-phenyl-N-(1-phenylpropyl)-4-quinolinecarboxamide.

16. The composition according to claim 13 wherein the active agent is Ropirinole, Paroxetine, or Kytril.

17. The composition according to claim 1 which is intended for oral administration.

18. The composition according to claim 1 in which the active agent demonstrates improved bioavailability.

19. The composition according to claim 1 in which the electrospun fiber is encapsulated or compressed into a tablet.

20. The composition according to claim 1 in which the electrospun fiber is further ground.

21. The composition according to claim 1 which is results in a rapid dissolution of the fiber.

22. The composition according to claim 1 which results in controlled release, sustained release, or pulsatile release of the active agent.

23. The composition according to claim 1 which results in immediate release of the active agent.

24. A process for making an electrospun pharmaceutical composition comprising a pharmaceutically acceptable active agent which agent is sparingly water soluble or water insoluble, and a pharmaceutically acceptable polymeric carrier, which process comprises
a) making a solution of the active agent, and pharmaceutically acceptable polymeric carrier with a pharmaceutically acceptable solvent; and
b) electrospinning the solution of step (a) into a fiber.

25. The process according to claim 24 wherein the solvent is water miscible.

26. The process according to claim 24 wherein the solvent is water immisicible.

27. The composition according to claim 24 wherein the solution is mixture of one or more solvents.

28. The process according to claim 27 wherein the solvent is a mixture of water and a water miscible solvent.

29. The process according to claim 24 wherein the polymeric carrier is poly(ethylene oxide), polyvinyl alcohol, polyvinyl pyrrolidone, hyaluronic acid, alginates, carragenen, carboxymethyl cellulose sodium, hydroxyethyl cellulose, hydroxypropylcellulose, hydroxypropylmethyl cellulose, hydroxypropylmethyl cellulose phthalate, cellulose acetate phthalate, starch, hydroxyethyl starch, sodium starch glycolate, chitosan and its derivatives, albumen, gelatin, or collagen.

30. The process according to claim 24 wherein the polymeric carrier is polyvinyl acetate, methyl cellulose, ethylcellulose, noncrystalline cellulose, polyacrylates and its derivatives, poly(alpha-hydroxy acids) and its copolymers such poly(caprolactone), poly(lactide-co-glycolide), poly(alpha-aminoacids) and its copolymers, poly(orthoesters), polyphosphazenes, poly(phosphoesters), or polyanhydrides.

31. The process according to claim 24 wherein the active agent is an analgesic, anti-inflammatory agent, anthelmintic, anti-arrhythmic agent, an antibiotic, anticoagulant, antidepressant, antidiabetic agent, antiepileptic, antihistamine, antihypertensive agent, antimuscarinic agent, antimycobacterial agent, antineoplastic agent, immunosuppressant, antithyroid agent, antiviral agent, anxiolytic sedative, astringent, beta-adrenoceptor blocking agent, contrast media, corticosteroid, cough suppressant, diuretic, dopaminergic, homeostatic, immunological agent, lipid regulating agent, muscle relaxant, parasympathomimetic, parathyroid, calcitonin, prostaglandin, radio-pharmaceutical, sex hormone, steroid, anti-allergic agent, antihistaminic, stimulant, sympathomimetic, thyroid agent, vasodilator, PDE IV inhibitor, or a mixture thereof.

32. The process according to claim 24 wherein the active agent is an anti-inflammatory agent, a PDE IV inhibitor, nabumetone, aspirin, cis-4-Cyano-4-[3-cyclopentyloxyl)-4-methoxyphenyl]-cyclohexanecarboxylic acid, or (S)-3-Hydroxy-2-phenyl-N-(1-phenylpropyl)-4-quinolinecarboxamide, Kytril, Zofran, Paroxetine, Ariflo, or Requip.

32. The process according to claim 24 wherein the active agent is water insoluble.

33. The process according to claim 24 wherein the active agent is sparingly water soluble.

34. The product produced by the process according to any one of claims 24 to 33.

35. A process for making an electrospun pharmaceutical composition comprising a pharmaceutically acceptable active agent wherein the agent is sparingly water soluble or water insouble, and a pharmaceutically acceptable polymeric carrier, which process comprises
a) melting the active agent and polymeric carrier; and
b) electrospinning the melt of step (a) into a fiber.

36. The process according to claim 35 wherein the polymeric carrier is poly(ethylene oxide), polyvinyl alcohol, polyvinyl pyrrolidone, hyaluronic acid, alginates, carragenen, carboxymethyl cellulose sodium, hydroxyethyl cellulose, hydroxypropylcellulose, hydroxypropylmethyl cellulose, hydroxypropylmethyl cellulose phthalate, cellulose acetate phthalate, starch, hydroxyethyl starch, sodium starch glycolate, chitosan and its derivatives, albumen, gelatin, or collagen.

37. The process according to claim 35 wherein the polymeric carrier is polyvinyl acetate, methyl cellulose, ethylcellulose, noncrystalline cellulose, polyacrylates and its derivatives, poly(alpha-hydroxy acids) and its copolymers such poly(caprolactone), poly(lactide-co-glycolide), poly(alpha-aminoacids) and its copolymers, poly(orthoesters), polyphosphazenes, poly(phosphoesters), or polyanhydrides.

38. The process according to claim 35 wherein the active agent is an analgesic, anti-inflammatory agent, anthelmintic, anti-arrhythmic agent, an antibiotic, anticoagulant, antidepressant, antidiabetic agent, antiepileptic, antihistamine, antihypertensive agent, antimuscarinic agent, antimycobacterial agent, antineoplastic agent, immunosuppressant, antithyroid agent, antiviral agent, anxiolytic sedative, astringent, beta-adrenoceptor blocking agent, contrast media, corticosteroid, cough suppressant, diuretic, dopaminergic, homeostatic, immunological agent, lipid regulating agent, muscle relaxant, parasympathomimetic, parathyroid, calcitonin, prostaglandin, radio-pharmaceutical, sex hormone, steroid, anti-allergic agent, antihistaminic, stimulant, sympathomimetic, thyroid agent, vasodilator, PDE IV inhibitor, or a mixture thereof.

39. The composition according to claim 35 wherein the active agent is an anti-inflammatory agent, a PDE IV inhibitor, nabumetone, aspirin, cis-4-Cyano-4-[3-cyclopentyloxyl)-4-methoxyphenyl]-cyclohexanecarboxylic acid, or (S)-3-Hydroxy-2-phenyl-N-(1-phenylpropyl)-4-quinolinecarboxamide, Kytril, Zofran, Paroxetine, Ariflo, or Requip.

40. The process according to claim 35 wherein the active agent is water insoluble.

41. The process according to claim 35 wherein the active agent is sparingly water soluble.

42. The product produced by the process according to any one of claims 35 to 41.

43. Use of a composition according to claim 1 for inhalation therapy.

44. Use of a composition according to claim 1 for dispersion in an aqueous solution.

45. The process according to claim 35 wherein the active agent is homogenously dispersed with the carrier in the fiber.
